(19) United States
(12) Patent Application Publication
(10) Pub. No.: US 2020/0022564 A1
(43) Pub. Date: Jan. 23, 2020

(54) DIRECT VIEW OPTICAL CARDIAC CATHETER

(71) Applicant: Edward A. HLOZEK, Somerville, MA (US)

(72) Inventor: Edward A. HLOZEK, Somerville, MA (US)

(21) Appl. No.: 16/531,481

(22) Filed: Aug. 5, 2019

Related U.S. Application Data
(63) Continuation of application No. 14/734,631, filed on Jun. 9, 2015, now abandoned.
(60) Provisional application No. 62/010,348, filed on Jun. 10, 2014.

(51) Int. Cl. A61B 1/00; A61M 25/00; A61B 5/00; A61M 25/01; A61B 1/018; A61B 1/05; A61B 1/06; A61B 5/042; A61B 18/14; A61B 18/24

(52) U.S. Cl. CPC A61B 1/00082; A61M 25/0053; A61B 5/0033; A61M 25/01; A61B 1/00045; A61B 1/018; A61B 18/082; A61B 1/06; A61B 5/042; A61B 18/1492; A61B 18/24; A61B 2560/04; A61B 1/051

(57) ABSTRACT

A device, a system, and a method for diagnosis and treatment of e.g., mitral valve regurgitation. The system includes a catheter that is configured to deflect and/or steer a distal tip of the catheter inside a patient's body, a guide wire that is configured to guide the catheter in an enclosed in vivo space within the patient's body, a proximal device handle that is configured to allow the non-parallel spiral cable to switch back and forth between flexibility modes, and a treatment device that is independent of, yet still within, an instrument channel of the catheter, wherein the distal tip of the catheter further includes an inflatable balloon which may be an asymmetrical intussuscepted shape, and may be attached within a section of the distal tip of the catheter, wherein the distal tip further includes a visualization device for directly viewing an in vivo space within the patient.

### CROSS-REFERENCE TO PRIOR APPLICATION

This application claims priority to and the benefit thereof from U.S. Provisional Patent Application No. 62/010,348, filed on Jun. 10, 2014, titled “Direct View Optical Catheter,” the entirety of which is hereby incorporated herein by reference.

### FIELD OF THE DISCLOSURE

The present disclosure relates generally to a device, a system, and a method for diagnosis and/or treatment of mitral valve regurgitation, including accurately positioning, for example, a cardiac catheter for diagnosing and/or treating mitral valve regurgitation.

### BACKGROUND OF THE DISCLOSURE

The heart is a four-chambered pump that moves blood efficiently through the cardiovascular system. Oxygen deprived blood enters the heart through the vena cava and flows into the right atrium. From the right atrium, blood flows through the tricuspid valve and into the right ventricle, which then contracts and forces blood through the pulmonic valve and into the pulmonary arteries leading to the lungs, where the blood is oxygenated and returned to the heart through the pulmonary veins. Gas exchange takes place in the lungs, whereby CO₂ is released from the blood and oxygen is absorbed. The oxygenated blood then returns from the lungs and enters the heart through the left atrium and passes through the bicuspid mitral valve into the left ventricle. The left ventricle contracts and pumps blood through the aortic valve into the aorta and to rest of the cardiovascular system. The cardiovascular system includes pulmonary and systemic circuits that function simultaneously.

The mitral valve comprises two valve leaflets (anterior and posterior) attached to a fibrous ring or annulus. In a healthy heart, the mitral valve leaflets overlap during contraction of the left ventricle and prevent blood from flowing back into the left atrium. An imperfect closure of the mitral valve can cause mitral regurgitation, or leakage, which is the backflow of the blood from the left ventricle into the left atrium.

It is a common medical practice to treat mitral valve regurgitation by valve replacement or repair. Both procedures involve an open-heart surgical procedure in which the patient's mitral valve is removed and replaced with an artificial valve. This is a complex, invasive surgical procedure with the potential for many complications and a long recovery period. For example, any time a patient undergoes open-heart surgery, there is a risk of infection. Opening the sternum and using a cardiopulmonary bypass machine has also been shown to result in a significant incidence of both short and long term neurological deficits.

A variety of devices, methods, and systems currently exist for treating mitral valve regurgitation. Of such devices, many are directed to open surgical techniques as well as complex endoscopic techniques that can be difficult to perform.

Some minimally invasive procedures have been developed to treat mitral valve regurgitation, but, to date, none have become commercially successful standard procedures. Furthermore, none of the known procedures provide a surgeon with a relatively clear visual view of the treatment site. Rather, the surgeon's view is obstructed by the blood within which the device is immersed and travels to the target site.

U.S. Pat. No. 6,619,291 to Hlavka et al. appears to disclose a minimally invasive method of performing annuloplasty including inserting an implant into a left ventricle and orienting the implant in the left ventricle substantially below the mitral valve. The implant and tissue around the mitral valve appear to be connected and tension appears to be provided to the implant in order to substantially reduce an arc length associated with the mitral valve.

In U.S. Pat. No. 6,718,985 and 7,037,334 to Hlavka et al. a series of plications near the mitral valve appear to be created by T-bars that are threaded together to reshape the mitral valve.

In U.S. Pat. No. 7,166,127 a catheter-based system for treatment of mitral valve regurgitation appears to use retainers adapted to be secured to the annulus of the mitral valve with flexible tensile members coupled to the retainers. A crimping device deployable through the catheter appears to compress a crimp onto the flexible tensile members after they are pulled toward one another to reduce the circumferential length of the annulus.

U.S. Pat. No. 8,197,464 appears to disclose a deflection guide catheter for use in minimally invasive medical procedures for plication of the mitral valve annulus where the appropriateness of the plication may be examined using imaging means such as TEE, ICE, TTE or fluoroscopy.

U.S. Pat. No. 8,252,006 appears to disclose a device for reducing the size of the stomach having a corkscrew-shaped anchor for placement in the gastric wall.

U.S. Pat. No. 7,599,747 appears to disclose a screw-in electrode probe having a corkscrew-shaped distal tip for use in cardiology applications.

In United States Patent Application Publication No. 2007/0093857, Rogers et al. appears describe a device and method for the treatment of mitral valve regurgitation using a minimally invasive procedure in which plications may be made proximate the mitral valve of the patient and a retainer is placed to hold the plication. The visualization of the target region may be done under fluoroscopy, ultrasound, or magnetic resonance imaging.

U.S. Pat. No. 8,394,015 to Christopher DiBiasio et al. discloses an instrument port for minimally invasive cardiac surgery. The patent appears to describe an instrument port for introducing instruments into a surgical site, including a port body having a channel running there-through from a proximal end to a distal end, an instrument sleeve in slidable contact with the channel.

Some minimally invasive procedures have been developed to treat mitral valve regurgitation, but, to date, none have become commercially successful standard procedures. Furthermore, none of the known procedures provide a surgeon with a relatively clear visual view of the treatment site. Rather, the surgeon's view is obstructed by the blood within which the device is immersed and travels to the target site.

There is an unfulfilled need for a device, as system and a method for diagnosing and/or treating mitral valve regurgitation that can be used efficiently and effectively in a minimally invasive procedure and that provides the surgeon with a direct and relatively unobstructed view of the target site, so that the procedure can be carried out accurately.

### SUMMARY OF THE DISCLOSURE

The present disclosure provides a device, a system, and a method for, but not limited to diagnosis and/or treatment of mitral valve regurgitation.

According to an aspect of the disclosure, a device is provided for treating damaged or deformed tissue, the device comprising: a casing that is configured to contact a tissue surface and conform to the shape of the tissue surface to provide a substantially clear view of the contacted tissue surface; and a visualization device that is configured to capture an image of the contacted tissue surface. The device may comprise an illumination device that illuminates the tissue surface.

The casing may have a intussuscepted shape to allow for a treatment device to pass through a central portion of the casing to treat a portion of the tissue.

The treatment device may comprise at least one of: grasping forceps; a laser beam transmitter; a scissor; a knife; a heating element; a radio frequency (RF) transducer; an electrode; or a cannula.

The casing may include a casing body having a clear chemical polymer that includes at least one electrode. The at least one electrode may sense biometric data related to the tissue. The at least one electrode may be configured to emit radiant energy to the tissue.

The visualization device may comprise at least one of: an optical camera chip; a charge coupled device (CCD) imaging chip; a CMOS imaging chip; or a coherent fiber-optic bundle.

The casing may be attached to a distal end of the catheter. The casing may extend beyond the distal end of the catheter.

The casing may comprise a balloon.

According to a further aspect of the disclosure, a system is provided for diagnosing or treating damaged or deformed tissue. The system comprises: a catheter that is configured to be inserted into a blood vessel; a casing that is affixed to a distal end of the catheter, the casing being configured to contact a tissue surface and conform to the shape of the tissue surface to provide a substantially clear view of the contacted tissue surface; a visualization device that is configured to capture an image of the contacted tissue surface; and a display device that displays the captured image of the contacted surface. The system may further comprise a treatment device to treat a portion of the tissue.

The treatment device may comprise at least one of: grasping forceps; a laser beam transmitter; a scissor; a knife; a heating element; a radio frequency (RF) transducer; an electrode; or a cannula.

The casing may extend beyond the distal end of the catheter. The casing may have an intussuscepted shape to allow for a treatment device to pass through a central portion of the casing to treat a portion of the tissue. The casing may be a balloon. The casing may include a body having a clear chemical polymer that includes at least one electrode. The casing may be attached to a distal end of the catheter.

The catheter may comprise an illumination device that illuminates the tissue.

The disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as any person skilled in the art would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the disclosure. No attempt is made to show structural details of the disclosure in more detail than may be necessary for a fundamental understanding of the disclosure and the various ways in which it may be practiced. In the drawings:

FIG. 1 shows an example of a system for treatment of mitral valve regurgitation, in accordance with the principles of this disclosure.

FIG. 2 shows an example of a catheter end with a device for treatment of mitral valve regurgitation, in accordance with the principles of this disclosure.

FIG. 3 shows a side view of the catheter end with an example of a pear-shaped casing.

FIG. 4 shows a side view of the catheter end with an example of a sphere-shaped casing.

FIG. 5 shows an example of the catheter end with another example of a treatment device for treatment of mitral valve regurgitation, in accordance with the principles of this disclosure.

FIG. 6 shows a close-up view of the device for treatment of mitral valve regurgitation of FIG. 5.

FIG. 7A shows a non-limiting example of an application of the catheter, including, the catheter's movement in a blood vessel.

FIG. 7B shows an example of a process for diagnosing and/or treating a mitral valve regurgitation in a patient, according to principles of the disclosure.

FIG. 8 shows an example of a heart with a lead wire inserted through the inferior vena cava into the right atrium, according to the principles of the disclosure.

FIG. 9 shows an enlarged view of the right atrium of the heart with the lead wire inserted therein and in contact with the interatrial septum.

FIG. 10 shows an enlarged view of the left atrium with the guide wire inserted through the right atrium and atrial interatrial septum into the left atrium.

FIG. 11 shows an enlarged view of the right atrium with the catheter device being provided through the inferior vena cava into the right atrium along the guide wire, before being inserted through the interatrial septum.

FIG. 12 shows an enlarged view of the left atrium with the catheter emerging from the interatrial septum, along the guide wire.

FIG. 13 shows an enlarged view of the left atrium with the catheter being guided and positioned proximate a portion of the mitral valve, and the casing beginning to expand.

FIG. 14 shows an enlarged view of the left atrium with the casing in an expanded configuration and a treatment device beginning to extend from the catheter end.

FIG. 15 shows an enlarged view of the left atrium with the catheter being positioned proximate another portion of the mitral valve.

The present disclosure is further described in the detailed description that follows.

### DETAILED DESCRIPTION OF THE DISCLOSURE

According to one aspect of the present disclosure, a device is provided for diagnosis and/or treatment of mitral valve regurgitation. The device comprises a catheter and a casing. The catheter may include one or more cable channels. The catheter may further include at least one steerable non-parallel spiral cable designed to deflect and/or steer a tip of the catheter inside a patient's body. The spiral cable(s) may be positioned in the cable channel(s). The cable may be made of, for example, titanium, nitinol, steel, or other metal alloy, or synthetic material such as plastic, or the like.

The catheter may include a plurality of (e.g., three) steerable non-parallel spiral cables, as is known in the art. The non-parallel spiral cables may be controlled by, for example, thumb and/or finger actuation of a proximal device handle, to steer the cables, and to make the cables more flexible or rigid by applying tension or relaxing tension on the proximal device handle, depending on what a user (e.g., a clinical operator, a surgeon, a physician, or the like) prefers to utilize.

The user may also utilize the proximal device handle to allow the non-parallel spiral cable(s) to switch back and forth between flexibility modes, or by utilizing temperature sensitive irrigation within the catheter's steerable cable channels to allow for inherent rigid or soft capabilities. The proximal device handle may be a separate device that is connected to the catheter.

The catheter includes a distal tip that may include a rigid ring of plastic, metal or other appropriate material to hold the non-parallel spiral cable anchored at the distal tip. The casing may be configured to be filled with a gas (e.g., air), a liquid (e.g., saline solution), or a gas-liquid mixture. The casing may include a balloon. The casing may be affixed to or integrally formed at an end with the catheter. The other (or opposite) end of the casing is configured to expand. The casing comprises an expandable (e.g., fillable) clear body. The casing body may be made of a clear material such as, for example, a clear polymer. The casing may include a non-symmetrical tear-drop shape. The casing may include a clear, chemical polymer, which may include one or more sensors or electrodes that may sense biometric data and/or generate and/or radiate energy to the target tissue.

The catheter may further include one or more illumination devices to illuminate the target site. The illumination device may include, e.g., a light emitting diode (LED), a fiber-optic element, or the like.

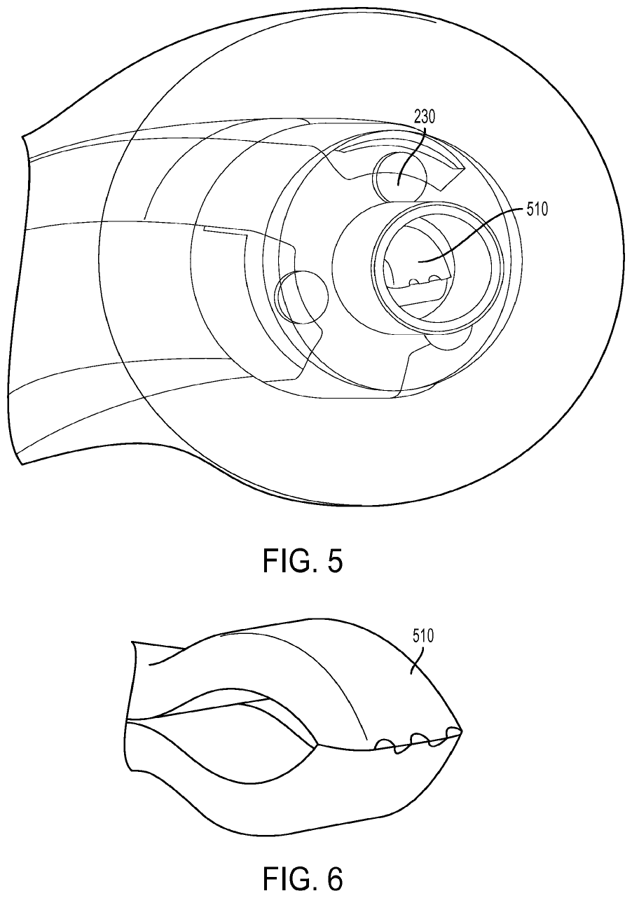

The casing may include a clear material to allow it to be pressed against the tissue and to thereby remove any naturally flowing opaque or non-translucent fluids (e.g., blood) from the field of view provided by the casing “window”. For instance, by applying pressure with the catheter against the tissue site via advancement of the catheter (or by expansion of the casing), fluids are squeezed away from the interface of the casing and the tissue to be viewed, thereby providing a “window” with a direct view of anatomical structures to be seen against the clear casing. The casing may include a visualization (or image pickup) device that may be positioned in, on or proximate to a rigid ring provided at the end of the catheter. For instance, the rigid ring may be positioned at the distal end of the catheter and the visualization device may be affixed within an opening of the rigid ring. Alternatively, the visualization device (or multiple visualization devices) may be affixed to a surface of the ring. FIG. 5 shows an example of a rigid ring that may be included in the catheter end.

The casing may include an intussuscepted shape around the distal tip of the catheter to allow for a through-put device to be independently inserted into the field of view of the optical components contained therein. The through-put device may include a treatment device, as described below.

The visualization device, which is enveloped in the casing body, may include e.g., an optical camera chip, a charge coupled device (CCD) imaging chip, a CMOS imaging chip, or the like, to be used to capture an image of a target, such as, e.g., anatomical structures, which can be transmitted to a computer that is located outside the patient's body, to display an image of the target on the computer's display. Alternatively, or additionally, the visualization device may include a coherent fiber-optic bundle that may convey the image through optics directly to a user using his/her naked eye. The image may be transmitted using a wired or a wireless communication link.

When shrunk (e.g., deflated or emptied), the casing may be of nominal size of the outer dimension of the catheter itself, and may also have a capacity to be expanded (e.g., inflated or filled) to full operational diameter to enhance utilization of “clearing a field of view”, in helping the user visualize the location of the catheter in an enclosed in vivo space within the patient. The casing may also be shrunk so as to retract to the nominal size of the outer dimension of the catheter itself. The casing body may include an “asymmetrical intussuscepted shape”.

The casing may be attached within, or integrally formed with a section of the distal tip of the catheter. The casing top of the catheter on the inner intussusception of the catheter may surround an instrument channel of nominal size (e.g., about 9 Fr in diameter) so as to allow passage of a treatment device that is independent of, yet still within, the instrument channel of the catheter itself, and without interfering with expanding and shrinking of the casing.

The treatment device may include e.g., grasping forceps, a laser beam transmitter, a scissor, a knife, a heating element, a radio frequency (RF) transducer, an electrode, a cannula, or the like. The treatment device may also include prosthetics or any attachment device such as, for example, staples or clips, which may fit through a channel within the catheter or treatment device. The treatment device may be designed to be passed over a standard guide wire through its instrument/through-put channel to gain access in the most minimally invasive method available to the user, and upon entry to the appropriate internal anatomical organ (e.g., heart), remain in place while the prior-placed guide wire is removed so as to allow for either irrigation or the treatment device to be placed within the instrument channel.

Another aspect of the present disclosure includes a method for diagnosing and/or treating mitral valve regurgitation. The method includes: administering an anesthetic to a patient (e.g., injecting a local anesthetic to a patient's tissue to numb a surrounding area of the skin); making an incision through the skin tissue and puncturing the femoral artery or vein; and inserting a guide wire into the arterial opening. A plastic sheath (with a stiffer plastic introducer inside it) may then be threaded over the wire and pushed into the artery. The wire may then be removed and the side-port of the sheath may be aspirated to ensure arterial blood flow back. The wire may then be flushed with saline. The arterial sheath, with a bleedback prevention valve, may then act as a conduit into the artery for the duration of the procedure.

A catheter, constructed according to the principles of the instant disclosure, may be inserted over the guide wire and moved towards the heart. Once in position above the aortic or mitral valve, the guide wire may be removed and the catheter may be positioned within, e.g., the left atrium, the left coronary artery, the right coronary artery, or the coronary sinus.

The disclosed method utilizes at least one visualization device located at a distal end of the catheter and enclosed in a casing to directly view a target site. The casing may include a balloon. According to an embodiment of the present disclosure, the catheter may include a plurality (e.g., three) of visualization devices at the distal end of the catheter.

The casing may be transparent and/or translucent and comprise, e.g., plastic or other transparent/translucent material. The casing may be pressed against a target site (e.g., tissue) to clear away any bodily fluids (e.g., blood) so that an image of the target site may be captured by the visualization device. The casing may be tear shaped and configured to be inflated/filled with gas (e.g., air) and/or fluid (e.g., saline solution). When deflated/emptied, the casing may be of nominal size of an outer dimension of the catheter so that it can travel within an enclosed in vivo space of the patient's arteries. The casing may be inflated/filled once it arrives at a target site in order to enhance the anatomical view of the target site, as seen, e.g., in FIG. 15.

The casing may have an “asymmetrical intussuscepted shape” which may surround an instrument channel of nominal size (e.g., about 9 Fr in diameter) so as to allow passage of a treatment device. The overall diameter of the treatment device may range from about 9 Fr to about 16 Fr. However, the diameter may be smaller than 9 Fr or larger than 16 Fr, as understood by one skilled in the art without departing from the scope or spirit of the disclosure. The treatment device may be independent of, yet still within an instrument channel of the catheter itself, and without interfering with expanding (e.g., inflating or filling) and shrinking (e.g., deflating or emptying) of the casing. The treatment device may include e.g., a forceps, a laser beam device, a knife, a scissor, an RF transducer, an electrode, a cannula, and the like. The treatment device may be connected to a controller device outside the patient's body. The controller device may allow the user to control the movement of the treatment device by e.g., inputting commands. The controller device may include a proximal device handle which may be controlled by a thumb and/or finger actuation of the proximal device handle, to steer the catheter, and to make it more flexible or rigid by applying tension or relaxing tension on the proximal device handle, depending on what a user (e.g., a clinical operator, a physician, and the like) prefers to utilize.

Once treatment of the mitral valve and concomitant reduction in mitral valve regurgitation is achieved, or other therapeutic applications, then the catheter may be withdrawn and the femoral access site may be closed using conventional closing techniques, such as, e.g., stitching.

In yet another embodiment of the present disclosure, the method for treatment of mitral valve regurgitation may further include a computer readable storage medium tangibly embodying computer readable program code having computer readable instructions which, when implemented, cause a computer to carry out the steps of the method disclosed above.

The casing may have e.g., a tear shape, a round shape, a rectangular shape, a triangular shape, a conical shape, and so on.

The system may further include a computer which may be configured to receive captured images over a communication link from the visualization device and display the images on a display. The computer may be located outside the patient's body.

The present disclosure provides a device, system and a method for in vivo diagnosing and treating, e.g., damaged or deformed tissue (e.g., mitral valve) by enabling a physician to maneuver and directly view the target site and perform in vivo diagnosis and treatment.

The disclosed device, method, and system provide new solutions, including, an ability to directly view the target site in vivo in a real-time, which will aid physicians in making critical medical decisions. The disclosed device, method, and system also allow steering and rigidity control of the treatment device within the patient's body. This provides physicians with an ability to examine, e.g., the mitral valve, during a procedure before taking an irreversible action. It will also reduce the need for, expense of, and risks associated with repeating medical procedures. The disclosed device, method, and system enable physicians to treat patients in a minimally invasive manner without an open-heart surgical procedure and without using a cardio bypass machine.

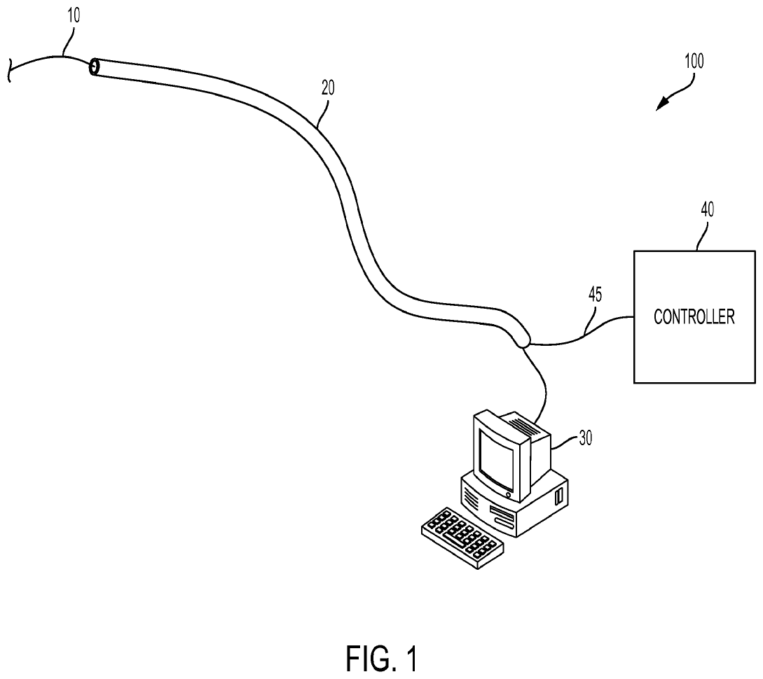

FIG. 1 shows an example of a system 100 that is constructed according to the principles of the disclosure. The system 100 is configured to provide a direct visual view of a target site in vivo and enable simultaneously treatment of tissue (such as, e.g., a mitral valve). The visualization of the target site and the treatment may both be done in real-time. The system 100 includes a guide wire 10, a catheter 20 which may be steered by the guide wire 10, a controller device 40, and a computer 30 which may also include e.g., a display device, all of which may be coupled to each other via communication links 45. For example, the computer 30 and the controller device 40 may be connected to each other and/or the catheter 20 via one or more communication links 45. The computer 30 may be used by a user, such as, e.g., a surgeon, a physician, an operator, a nurse, and the like.

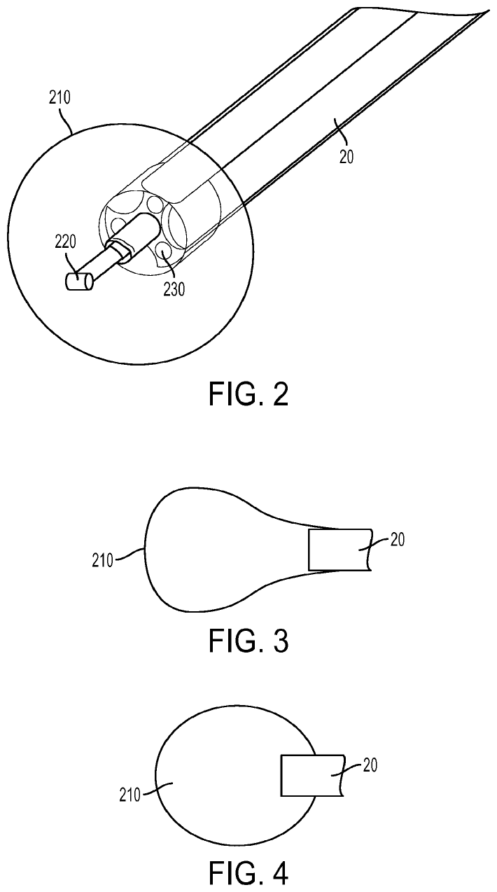

FIG. 2 shows an example of a catheter 20 that is constructed according to the principles of the disclosure. The catheter 20 may include an expandable casing 210, at least one visualization device 230, and a treatment device 220. The catheter 20 may include an illumination device 234 for projecting a light beam. The treatment device 220 may apply radiant (e.g., laser beam, ultrasound, etc.) or contact (e.g., heating electrode) energy, physical pressure, electricity, or the like. The treatment device 220 may include, e.g., an electrode assay. Unlike a typical cardiac catheterization procedure where x-ray opaque contrast agents are injected to enable the coronary vessels to appear on the x-ray fluoroscopy image to visualize the target area, the disclosed device utilizes at least one visualization device 230 located at a distal end (e.g., at the a tip) of the catheter 20 to view the target site in real time. The visualization device 230 is enclosed in the casing 210, which may be pressed against a target site (e.g., tissue, bone, etc.) to directly view the target site. The casing 210 may include a balloon. The visualization device 230 may include, as noted previously, a plurality of visual devices, including, e.g., a CCD imaging chip, CMOS imaging chip, a camera, or the like. In an embodiment of the present disclosure, the visualization device 230 may include three visualization devices 230 at the tip of the catheter 20.

Referring to FIGS. 1-2, the treatment device 220 may be connected to the controller 40. The controller 40 may enable the user to manipulate and control the movement of the treatment device 220 and/or catheter 20 by e.g., inputting commands. The controller 40 may include a proximal device handle (not shown) which may be controlled by a thumb and/or finger actuation of the proximal device handle, to steer the catheter, and to make it more flexible or rigid by applying tension or relaxing tension on the proximal device handle, depending on what the user prefers to utilize. The visualization device 230 may transmit captured images (via the communication link 45) to the computer 30 for e.g., viewing, storage, editing, or the like. The computer 30 may also control the catheter's movements inside the patient's body.

The casing 210 may be transparent or translucent and be comprised of e.g., plastic or other translucent material. The casing 210 may be pressed against a target site (e.g., tissue) to clear away any bodily fluids (e.g., blood) so that an image of the target site may be captured by the visualization device 230. The casing 210 is configured to expand and shrink. The casing 210 may be tear-shaped (or pear-shaped) and configured to be filled with a gas or a liquid. When shrunk, the casing 210 will be of nominal size of an outer dimension of the catheter 20 so that it can travel within an enclosed in vivo space of the patient's arteries. The casing 210 may be filled to expand to its full (or partially) full form once it arrives at a target site. The casing 210 may further surround the tip of the catheter 20 in an “asymmetrical intussuscepted shape” to provide an instrument channel of nominal size (e.g., 9 Fr in diameter) so as to allow passage of the treatment device 220 that is independent of, yet still within, the instrument channel of the catheter 20 itself, and without interfering with expanding (e.g., inflating or filling) and shrinking (e.g., deflating or emptying) of the casing 210. The treatment device 220 may include e.g., forceps, a transducer device (e.g., a laser transmitter, an RF transmitter, an electrode, an ultrasound transducer, etc.), a knife, a scissor, a cannula, and the like. The forceps 510 (shown in FIGS. 5 and 6) may be coupled to an RF transducer that generates and conveys energy to the forceps 510, which may be applied as RF energy to the target site.

The casing 210 may include a casing body having a clear chemical polymer that includes at least one electrode (not shown). The at least one electrode may sense biometric data related to the tissue, such as, for example, temperature, pressure, and the like. The at least one electrode may be configured to emit radiant energy to the tissue, such as, e.g., infrared energy, ultraviolet energy, x-ray energy, or the like.

FIG. 3 shows a side view of the catheter end with an example of a pear-shaped casing 210.

FIG. 4 shows a side view of the catheter end with an example of a sphere-shaped casing 210.

FIGS. 5-6 show one non-limiting example of the treatment device 220 inside the catheter 20. The treatment device 220 includes radio frequency (RF) grasping forceps 510.

FIG. 5 shows an example of the catheter 20 end with RF forceps 510 retracted within the catheter 20. The RF forceps 510 are configured to extend from the catheter for treating mitral valve regurgitation. As seen in FIG. 5, the treatment device 510 may extend outside of the ring at the end of the catheter 20.

FIG. 6 shows a close-up view of the forceps 510 in the catheter 20 of FIG. 5.

Referring to FIGS. 5 and 6, the radio frequency (RF) grasping forceps 510 may be configured to be in a closed position inside the catheter 20. The RF grasping forceps 510 may be configured to expand as they are moved out and away from the distal end of the catheter 20. Although seen as comprising serrations on the gripping ends of the forceps 510, the forceps may include smooth, substantially planar gripping surfaces. Further, the forceps 510 may be configured to retract and compress as they are drawn into the distal end of the catheter 20. The forceps 510 may be constructed of a compressible material (e.g., metal) that is normally in an open position (not shown), so that the two (or more) members of the forceps 510 may be forced to a closed position (shown in FIG. 6) by the inner walls of the catheter 20 or by the outer sheath (not shown) of the forceps 510, itself comprised of inner actuating members and an outer sleeve so as to be able to move forward/towards or backwards/away from an intended anatomical viewing area (target area) within the “window” desired, to achieve such desired positioning of the forceps jaws as needed, independent from any movement of the catheter 20 itself. Accordingly, the forceps 510 may be driven out from the catheter 20 or by the outer sheath of the forceps 510, itself comprised of inner actuating members and an outer sleeve so as to be able to move forward/towards or backwards/away from an intended anatomical viewing area within the “window” desired, to achieve such desired positioning of the forceps jaws as needed to grab a portion of target tissue, and then retracted into the catheter to cut, atraumatically grasp and hold the portion of the target tissue independent of any movement of the catheter 20 itself.

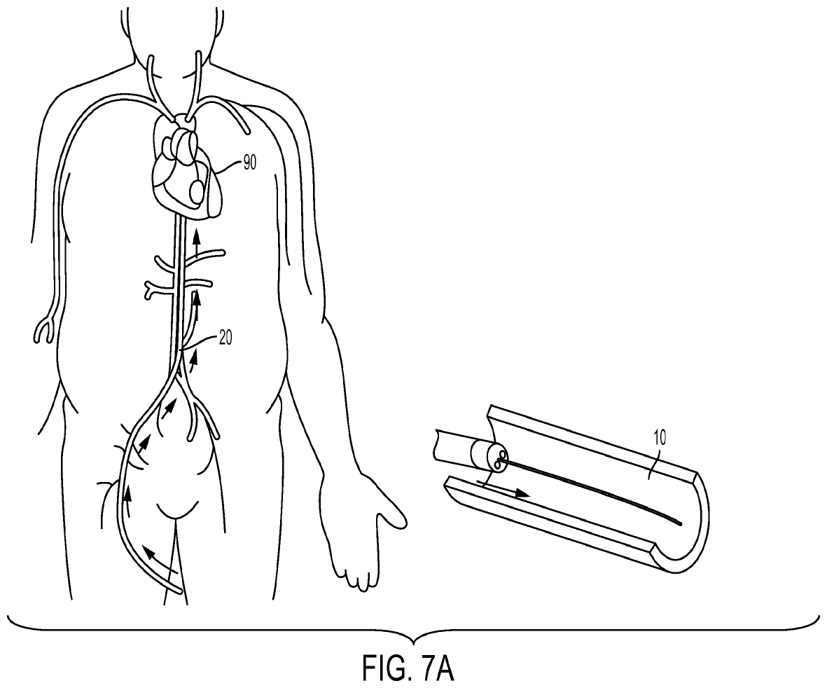

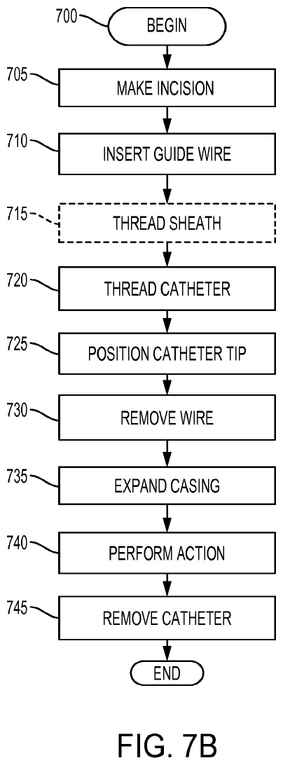

FIGS. 7A and 7B show non-limiting examples of a catheter's movement from a femoral artery into a heart and a process 700 for diagnosing and/or treating a mitral valve regurgitation in a patient.

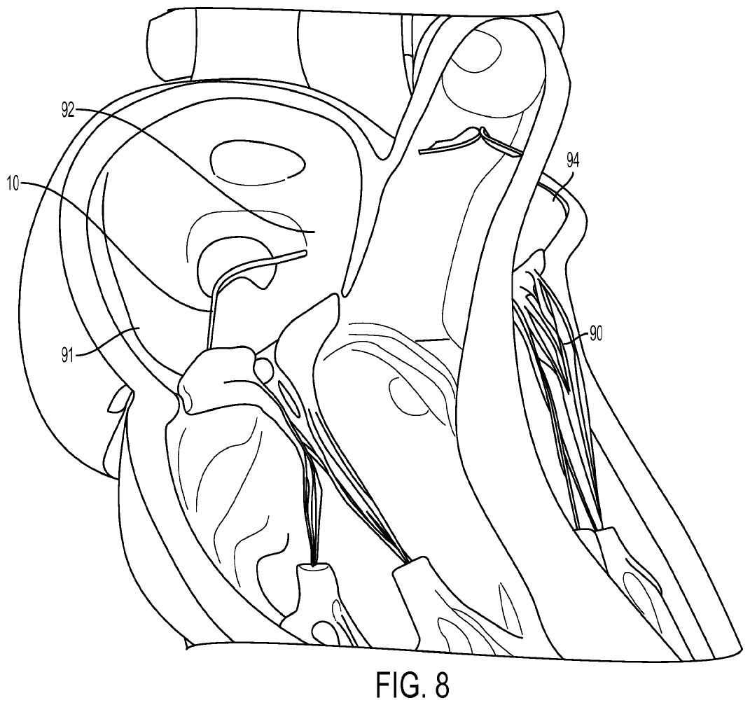

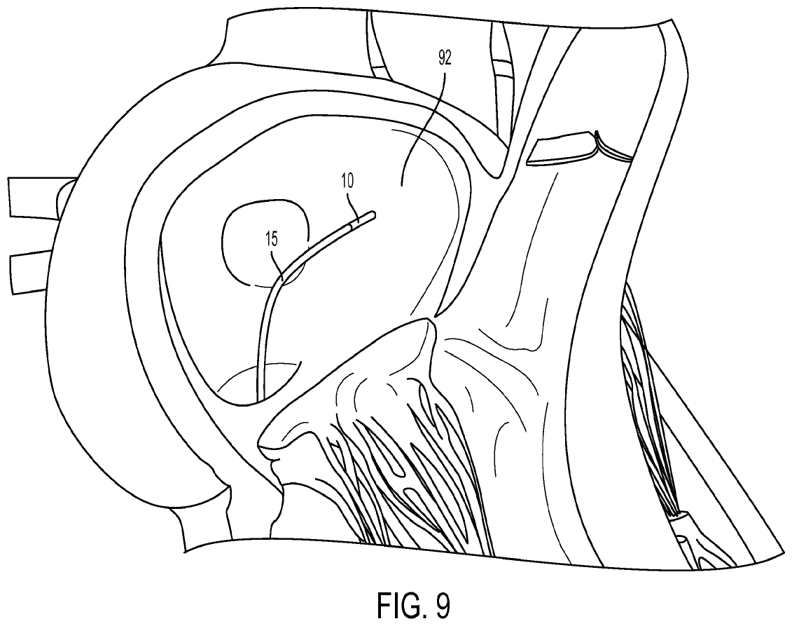

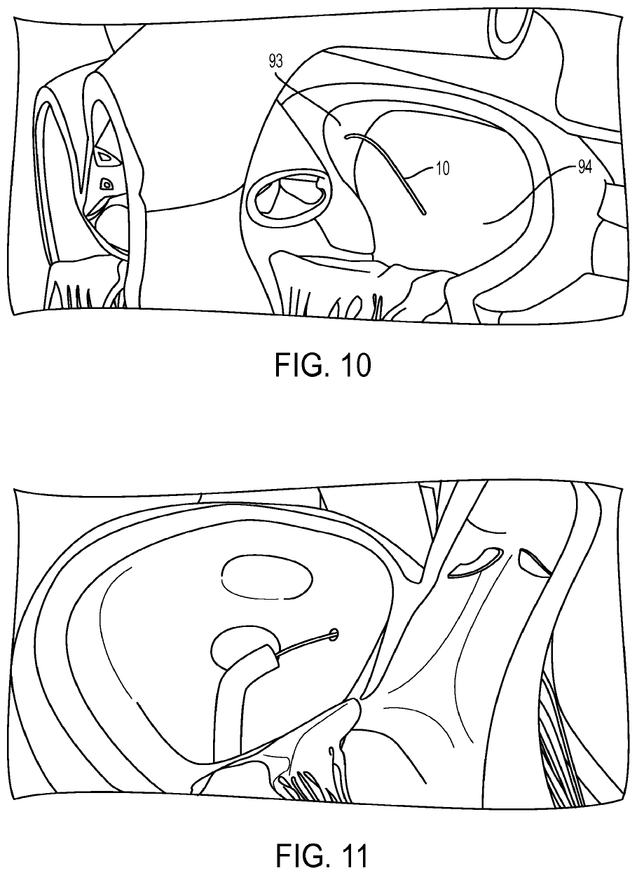

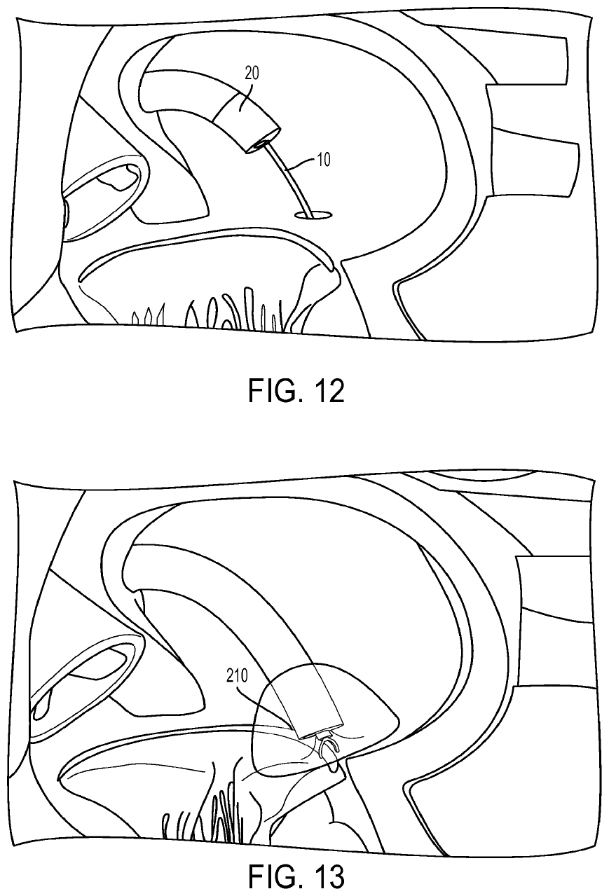

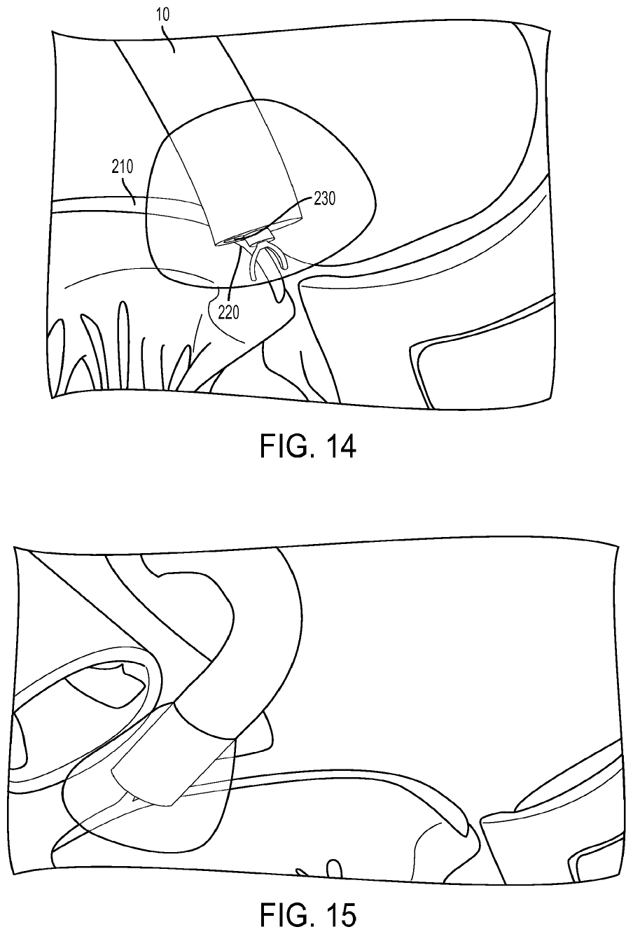

FIGS. 8 through 15 show various stages of the process 700: FIG. 8 shows an example of a heart 90 with the lead wire 10 inserted through the inferior vena cava into the right atrium 91; FIG. 9 shows an enlarged view of the right atrium 91 of the heart with the lead wire 10 inserted therein and in contact with the interatrial septum 92; FIG. 10 shows an enlarged view of the left atrium 94 with the guide wire inserted through the right atrium 91 and atrial interatrial septum 92 into the left atrium 94; FIG. 11 shows an enlarged view of the right atrium 91 with the catheter device 20 being provided through the inferior vena cava into the right atrium 91 along the guide wire 10, before being inserted through the interatrial septum 92; FIG. 12 shows an enlarged view of the left atrium 94 with the catheter 20 emerging from the interatrial septum 92, along the guide wire 10; FIG. 13 shows an enlarged view of the left atrium 94 with the catheter 20 being guided and positioned proximate a portion of the mitral valve 93, and the casing 210 beginning to expand; FIG. 14 shows an enlarged view of the left atrium 94 with the casing 210 in an expanded configuration and a treatment device 220 beginning to extend from the catheter 20 end, with the target area being imaged by the visualization device 230; and FIG. 15 shows an enlarged view of the left atrium 94 with the catheter 20 being positioned proximate another portion of the mitral valve 93.

Referring to FIGS. 7A, 7B and 8-15, the process 700 may begin after an anesthetic is administered to a patient, such as by, e.g., injecting a local anesthetic to the patient's tissue to numb a surrounding area of the skin. An incision may be made through the skin tissue, puncturing the femoral artery or vein (STEP 705). The guide wire 10 may be inserted into the arterial opening (STEP 710) and, optionally, a plastic sheath 15 (with a stiffer plastic introducer inside it) may be threaded over the wire 10 and pushed into and through the interatrial septum 92 (STEP 715).

The wire 10 may be removed and a side-port of the sheath 15 may be aspirated to ensure arterial blood flow back. The wire 10 may be flushed with saline. The sheath 15 may include a bleedback prevention valve (not shown). The sheath 15, with a bleedback prevention valve, may then act as a conduit into the artery (or vein) for the duration of the procedure.

A catheter 20, constructed according to the principles of the instant disclosure, may be inserted over the guide wire 10 and moved through, e.g., the right atrium 91, interatrial septum 92 and into the left atrium 94 (STEP 720). Once in position above the mitral valve 93 (STEP 725), the guide wire 10 may be removed (STEP 730) and the catheter 20 may be positioned proximate a treatment area on the target tissue. The casing 210 may then be expanded (e.g., filled with saline) (STEP 735) and images of the target site may be captured via the visualization device 230 and communicated to the computer 30. The casing 210 may be expanded and positioned such that substantially all fluid is removed between a portion of the casing body and the target tissue, thereby forming a “window” through which unobstructed images of the target tissue may be captured by means of the visualization device 230 and communicated to the computer 30. The casing 210 body may contact the target tissue such that it matches the contours of the target tissue, to minimize any fluid between the casing body and tissue surface.

As noted earlier, the casing 210 may be transparent and made of a material that, when pressed against a target site (e.g., tissue), substantially all bodily fluids (e.g., blood) will be forced away, so that a substantially unobstructed image of the target site may be captured by the visualization device 230. The casing 210 may have an “asymmetrical intussuscepted shape” which may surround the instrument channel, so as to allow passage of the treatment device 220 through the opening in the casing to the treatment site.

The treatment device 220 may be extended from the catheter and applied to the tissue site, such as, e.g., tissue surrounding the mitral valve 93 (shown in FIG. 14) to treat the target area (STEP 740). After repeating the steps of repositioning the catheter 20 tip and treating different portions of tissue to completion, the catheter 20 may be removed (STEP 745).

Once treatment (e.g., of the mitral valve and concomitant reduction in mitral valve regurgitation) is achieved, or other therapeutic applications, then the catheter 20 may be withdrawn and the femoral access site may be closed using conventional closing techniques, such as, e.g., stitching.

According to a further aspect of the disclosure, therapies may be delivered to target sites that can cross the casing's wall (e.g., polymer barrier) without affecting the integrity of the casing, such as, e.g., certain laser wavelengths.

It is contemplated that the device disclosed herein may be used in bodily organs or naturally existing collecting reservoirs that contain only translucent or clear fluids.

It is further contemplated that the computer 30 may be communicatively coupled to a network, so as to allow for, e.g., remote access, monitoring, and/or control.

It is further contemplated that the device and system disclosed herein may be used in other applications, such as, for example, treating articles or items that are submersed in fluids that obstruct image pickup.

A “computer,” as used in this disclosure, means any machine, device, circuit, component, or module, or any system of machines, devices, circuits, components, modules, or the like, which are capable of manipulating data according to one or more instructions, such as, for example, without limitation, a processor, a microprocessor, a central processing unit, a general purpose computer, a cloud, a super computer, a personal computer, a laptop computer, a palmtop computer, a notebook computer, a desktop computer, a workstation computer, a server, or the like, or an array of processors, microprocessors, central processing units, general purpose computers, super computers, personal computers, laptop computers, palmtop computers, notebook computers, desktop computers, workstation computers, servers, or the like.

A “communication link,” as used in this disclosure, means a wired and/or wireless medium that conveys data or information between at least two points. The wired or wireless medium may include, for example, a metallic conductor link, a radio frequency (RF) communication link, an Infrared (IR) communication link, an optical communication link, or the like, without limitation. The RF communication link may include, for example, WiFi, WiMAX, IEEE 802.11, DECT, 0G, 1G, 2G, 3G or 4G cellular standards, Bluetooth, and the like.

A “network,” as used in this disclosure means, but is not limited to, for example, at least one of a local area network (LAN), a wide area network (WAN), a metropolitan area network (MAN), a personal area network (PAN), a campus area network, a corporate area network, a global area network (GAN), a broadband area network (BAN), a cellular network, the Internet, the cloud network, or the like, or any combination of the foregoing, any of which may be configured to communicate data via a wireless and/or a wired communication medium. These networks may run a variety of protocols not limited to TCP/IP, IRC or HTTP.

The terms “including,” “comprising” and variations thereof, as used in this disclosure, mean “including, but not limited to,” unless expressly specified otherwise.

The terms “a,” “an,” and “the,” as used in this disclosure, means “one or more,” unless expressly specified otherwise.

Devices that are in communication with each other need not be in continuous communication with each other, unless expressly specified otherwise. In addition, devices that are in communication with each other may communicate directly or indirectly through one or more intermediaries.

Although process steps, method steps, algorithms, or the like, may be described in a sequential order, such processes, methods and algorithms may be configured to work in alternate orders. In other words, any sequence or order of steps that may be described does not necessarily indicate a requirement that the steps be performed in that order. The steps of the processes, methods or algorithms described herein may be performed in any order practical. Further, some steps may be performed simultaneously.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device may be alternatively embodied by one or more other devices which are not explicitly described as having such functionality or features.

Various forms of computer readable media may be involved in carrying sequences of instructions to a computer. For example, sequences of instruction (i) may be delivered from a RAM to a processor, (ii) may be carried over a wireless transmission medium, and/or (iii) may be formatted according to numerous formats, standards or protocols, including, for example, WiFi, WiMAX, IEEE 802.11, DECT, 0G, 1G, 2G, 3G or 4G cellular standards, Bluetooth, or the like.

While the disclosure has been described in terms of exemplary embodiments, those skilled in the art will recognize that the disclosure can be practiced with modifications in the spirit and scope of the appended claims. These examples are merely illustrative and are not meant to be an exhaustive list of all possible designs, embodiments, applications or modifications of the disclosure.

For instance, the instant disclosure may be used in applications that are outside of the medical field. The device, system and method of the present disclosure may be used in just about any applications where it is desirable to view, assess and treat a target site in an environment wherein the field of view is obstructed by moving or movable particles.

What is claimed:

1. A device for treating damaged or deformed tissue, the device comprising:
a casing that is configured to contact a tissue surface and conform to the shape of the tissue surface to provide a substantially clear view of the contacted tissue surface; and
a visualization device that is configured to capture an image of the contacted tissue surface.

2. The device according to claim 1, wherein the casing has an intussuscepted shape to allow for a treatment device to pass through a central portion of the casing to treat a portion of the tissue.

3. The device according to claim 1, further comprising:
an illumination device that illuminates the tissue surface.

4. The system according to claim 2, wherein the treatment device comprises at least one of:
grasping forceps;
a laser beam transmitter;
a scissor;
a knife;
a heating element;
a radio frequency (RF) transducer;
an electrode; or
a cannula.

5. The device according to claim 1, wherein the casing comprises a body having a clear chemical polymer that includes at least one electrode.

6. The device according to claim 5, wherein the at least one electrode senses biometric data related to the tissue.

7. The device according to claim 5, wherein the at least one electrode emits radiant energy to the tissue.

8. The device according to claim 1, wherein the visualization device comprises at least one of:
an optical camera chip;
a charge coupled device (CCD) imaging chip;
a CMOS imaging chip; or
a coherent fiber-optic bundle.

9. The device according to claim 1, wherein the casing is attached to a distal end of the catheter.

10. The device according to claim 9, wherein the casing extends beyond the distal end of the catheter.

11. The device according to claim 1, wherein the casing comprises a balloon.

12. A system for diagnosing or treating damaged or deformed tissue, the system comprising:
a catheter that is configured to be inserted into a blood vessel;
a casing that is affixed to a distal end of the catheter, the casing being configured to contact a tissue surface and conform to the shape of the tissue surface to provide a substantially clear view of the contacted tissue surface;
a visualization device that is configured to capture an image of the contacted tissue surface; and
a display device that displays the captured image of the contacted surface.

13. The system according to claim 12, further comprising:
a treatment device to treat a portion of the tissue.

14. The system according to claim 13, wherein the treatment device comprises at least one of:
grasping forceps;
a laser beam transmitter;
a scissor;
a knife;
a heating element;
a radio frequency (RF) transducer;
an electrode; or
a cannula.

15. The system according to claim 12, wherein the casing extends beyond the distal end of the catheter.

16. The system according to claim 12, wherein the casing has an intussuscepted shape to allow for a treatment device to pass through a central portion of the casing to treat a portion of the tissue.

17. The system according to claim 12, wherein the catheter comprises an illumination device that illuminates the tissue.

18. The system according to claim 12, wherein the casing comprises a body having a clear chemical polymer that includes at least one electrode.

19. The system according to claim 12, wherein the casing is attached to a distal end of the catheter.

20. The system according to claim 12, wherein the casing comprises a balloon.
